(19) United States
(12) Patent Application Publication
(10) Pub. No.: US 2008/0070230 A1
(43) Pub. Date: Mar. 20, 2008

(54) Methods for reducing or preventing liquid-liquid phase separation in high concentration protein solutions

(75) Inventors: Li Li, Sudbury, MA (US); Angela Kantor, Pepperell, MA (US); Nicholas W. Warne, Andover, MA (US)

Correspondence Address: WilmerHale/Wyeth, 60 STATE STREET, BOSTON, MA, 02109, US

(73) Assignee: Wyeth, Madison, NJ (US)

(21) Appl. No.: 11/811,517

(22) Filed: Jun. 11, 2007

Related U.S. Application Data
(60) Provisional application No. 60/812,760, filed on Jun. 12, 2006.

(51) Int. Cl. G01N 33/536; C07K 1/00; G01N 33/00

(52) U.S. Cl. 435/4; 436/536; 530/412

(57) ABSTRACT

Methods of formulating proteins at high concentrations in protein solutions, wherein the protein solutions lack or have reduced liquid-liquid phase separation are described. Such protein solutions are substantially clear and substantially homogenous. Additionally, methods of concentrating and purifying proteins using liquid-liquid phase separation are provided.

### CROSS REFERENCE TO RELATED APPLICATIONS

This application claims the benefit of U.S. Provisional Appl. No. 60/812,760, filed Jun. 12, 2006, the contents of which are incorporated herein by reference.

### FIELD OF THE INVENTION

The present invention relates generally to the field of protein formulations.

### BACKGROUND OF THE INVENTION

The availability of biotechnology-derived protein and peptide drugs has created new options for the treatment and prevention of numerous diseases. Protein-based therapy, especially monoclonal antibody-based therapy, has become an important method to treat diseases such as cancer, allergic diseases, asthma, and rheumatoid arthritis.

Antibody-based therapy is typically administered to a patient at regular intervals, and requires several mg/kg dosing by injection. Subcutaneous injection is a preferred route of administration of these therapies. Because of the small volumes used for subcutaneous injection (usually 1.5 mL), for high dose antibody therapies, this route of administration requires high concentration protein formulations.

One of the challenges in using high concentration protein solutions is the development of opalescence (i.e., cloudiness) in protein solutions above a particular protein concentration. This phenomenon can create non-homogeneous solutions and affect processing of the protein solution. Furthermore, opalescence of a solution may have negative commercial consequences. Thus, it is desirable to obtain a clear protein solution for ease of handling during processing, and for a more appealing appearance of the final drug product.

Accordingly, there is a need to develop methods to obtain high concentration protein solutions that are not opalescent (i.e., cloudy).

### SUMMARY OF THE INVENTION

The invention relates to methods for formulating high concentration protein solutions. More specifically, the invention relates to methods for reducing or preventing liquid-liquid phase separation of protein solutions formulated at high concentrations of the protein. Thus, the invention generally provides high concentration protein solutions that have reduced opalescence and therefore are visibly clear. As discussed herein, a visibly clear solution facilitates the ease of handling during processing, and is more appealing as a commercialized product. In addition, the invention provides methods of concentrating and purifying proteins using liquid-liquid phase separation of protein solutions.

In one aspect, the invention provides methods for determining a concentration at which to reformulate a protein in a protein solution that exhibits liquid-liquid phase separation so as to reduce or prevent liquid-liquid phase separation of the protein solution. More specifically, methods for reducing and/or preventing liquid-liquid phase separation includes maintaining or adjusting the solution to a desired temperature. For example, one method comprises allowing the protein solution that exhibits liquid-liquid phase separation to undergo liquid-liquid phase separation into upper and lower liquid phases. The concentration of the protein in the upper and lower phase of the protein solution that has undergone liquid-liquid phase separation is then measured. A protein concentration that is greater than the protein concentration of the lower phase of the protein solution is chosen to reformulate the protein. Formulating a protein solution using a concentration determined in this manner reduces or prevents liquid-liquid phase separation in the protein solution at the desired temperature. In certain embodiments, the protein concentration at which to formulate a protein in a protein solution at a desired temperature is selected to be about 0.5% to about 40% higher than the concentration of the protein in the lower phase. In other embodiments, the protein concentration at which to formulate a protein in a protein solution at a desired temperature is selected to be about 0.5% to about 5%, about 5% to about 10%, about 10% to about 20%, about 20% to about 30%, about 30% to about 40%, about 5% to about 20%, about 5% to about 30%, or about 5% to about 40% higher than the concentration of the protein in the lower phase.

In other aspects of the invention, methods for determining a protein concentration at which to reformulate a protein in a protein solution that exhibits liquid-liquid phase separation so as to reduce or prevent liquid-liquid phase separation of the protein solution at a desired temperature include constructing a phase separation curve for the protein solution, and choosing a concentration that is outside the phase separation curve at the desired temperature. A concentration that is outside the phase separation curve at the desired temperature permits reformulation of the protein in the protein solution so as to reduce or prevent liquid-liquid phase separation in the protein solution at the desired temperature. In certain embodiments of this aspect, the phase separation curve is constructed by plotting the concentration of the two coexisting liquid phases in equilibrium of the protein solution at two or more different temperatures at which the protein solution exhibits liquid-liquid phase separation. In certain embodiments, at least one of the two or more different temperatures is the desired temperature.

In a further aspect of the invention, methods for determining a concentration at which to formulate a protein in a protein solution to reduce or prevent liquid-liquid phase separation in the protein solution at a desired temperature include providing at least a first protein solution having a protein concentration and a first temperature at which the first protein solution exhibits liquid-liquid phase separation, and at least a second protein solution at the concentration of the first protein solution and a second temperature at which the second protein solution exhibits liquid-liquid phase separation. These methods further involve allowing the first and second protein solutions to undergo liquid-liquid phase separation into an upper and a lower phase. The concentration of the proteins in the upper and lower phases of the first and second protein solutions is measured and used to construct a phase separation curve. This phase separation curve specifies the concentration of the upper and lower phases in equilibrium of the first and second protein solutions at the first and second temperatures. A protein concentration that is outside the phase separation curve at the desired temperature is chosen to formulate the protein in the protein solution so as to reduce or prevent liquid-liquid phase separation in the protein solution at the desired temperature. In certain embodiments of this aspect, a third protein solution having the same protein concentration of the first protein solution and a third temperature is allowed to undergo liquid-liquid phase separation and the concentrations of its upper and lower layers are included in constructing the phase separation curve. In other embodiments, a fourth protein solution having the same protein concentration of the first protein solution and a fourth temperature is allowed to undergo liquid-liquid phase separation and the concentrations of its upper and lower layers are included in constructing the phase separation curve. In another embodiment, a fifth protein solution having the same protein concentration of the first protein solution and a fifth temperature is allowed to undergo liquid-liquid phase separation and the concentrations of its upper and lower layers are included in constructing the phase separation curve. As can be appreciated by the teachings herein, additional protein solutions can be included in constructing the phase separation curve using the methods described herein.

In another aspect of the invention, a method for determining a concentration at which to formulate a protein in a protein solution so as to reduce or prevent liquid-liquid phase separation in the protein solution at a desired temperature include providing the protein in a first protein solution at a first concentration, providing the protein in a second protein solution at a second concentration, and providing the protein in a third protein solution at a third concentration, wherein the first, second, and third protein solutions are at a temperature wherein the first, second, and third solutions do not exhibit liquid-liquid phase separation. The methods further comprise cooling the first, second, and third solutions to the cloud-point temperature of the first, second, and third solutions, and constructing a phase separation curve by plotting the cloud-point temperatures of the first, second, and third protein solutions against the first, second, and third concentrations. A concentration that is outside the phase separation curve at the desired temperature is determined to be the concentration at which to formulate the protein in the protein solution so as to reduce or prevent liquid-liquid phase separation in the protein solution at the desired temperature.

In another aspect of the invention, methods for formulating a protein in a protein solution wherein the protein solution does not exhibit liquid-liquid phase separation at a desired temperature include formulating the protein in the protein solution at a concentration that is outside a phase separation curve for the protein solution at the desired temperature.

In yet another aspect of the invention, methods for reformulating a protein in a protein solution that exhibits liquid-liquid phase separation at a desired temperature wherein the reformulated protein solution does not exhibit liquid-liquid phase separation at the desired temperature include subjecting the protein solution that exhibits liquid-liquid phase separation to undergo liquid-liquid phase separation into an upper and a lower liquid phase. The concentration of the upper and the lower phases of the protein solution is then measured. The protein is reformulated in a solution at a concentration that is higher than the concentration of the lower phase of the protein solution. This reduces or prevents liquid-liquid phase separation in the reformulated protein solution at the desired temperature.

In a still further aspect of the invention, methods for formulating a protein in a protein solution so that the protein solution does not exhibit liquid-liquid phase separation at a desired concentration include storing the protein solution at a temperature that is above the temperature at which liquid-liquid phase separation occurs and which is outside a phase separation curve for the protein solution.

In another aspect of the invention, methods for obtaining a protein solution that does not exhibit liquid-liquid phase separation at a desired temperature from a protein solution that exhibits liquid-liquid phase separation at the desired temperature includes allowing the protein solution that exhibits liquid-liquid phase separation to undergo separation into upper and lower phases at the desired temperature. The lower phase of the protein solution is removed. This lower phase is a protein solution that does not exhibit liquid-liquid phase separation at the desired temperature.

The invention further provides methods for obtaining a higher concentration protein solution from a lower concentration protein solution that exhibits liquid-liquid phase separation. The methods generally comprise allowing the original protein solution to separate into upper and lower phases, and separating the lower phase from the upper phase. The lower phase is a protein solution having a higher concentration of the protein than the lower concentration protein solution.

In another aspect of the invention, a method for concentrating a protein in a desired solution include formulating the protein in any solution, wherein the protein solution exhibits liquid-liquid phase separation. Liquid-liquid phase separation is facilitated in the protein solution, thereby forming an upper and lower phase in the protein solution. The lower phase is separated from the upper phase. This lower phase has a higher concentration of the protein than the original protein solution. A buffer exchange is then conducted to transfer the protein from the protein solution it is in into the desired solution.

“Protein” as used herein includes proteins, peptides, protein fragments, conjugated proteins, and polypeptides that contain non-naturally-occurring amino acids. A protein according to the invention can be a receptor, a ligand, a transcription factor, an enzyme, a coagulation factor, a signaling protein, and an antibody. Proteins can also be a polyclonal antibody or an antigen-binding fragment thereof, or a monoclonal antibody or an antigen-binding fragment thereof.

The desired temperature for a protein solution can be the temperature at which the protein solution is to be processed, manufactured, stored, or administered. Furthermore, liquid-liquid phase separation can be achieved by gravity separation or centrifugation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the drawings, detailed description, and the claims.

### BRIEF DESCRIPTION OF THE DRAWINGS

FIG. 1 is a graph depicting the inverse relationship between relative solubility of a protein as measured by PEG precipitation and opalescence as measured by absorbance at 500 nm. All proteins in this experiment were formulated at 90 mg/ml in 10 mM Histidine at pH 6.0.

FIG. 2 is an illustration of the phenomenon of liquid-liquid phase separation using anti-Al antibody (70 mg/ml in 10 mM Tris pH 8.0 at 5° C.) and anti-B1 monoclonal antibody (50 mg/ml in 20 mM succinate buffer pH 6.0 at 5° C.). Liquid-liquid phase separation is observed in the tubes on the left.

FIG. 3 is a schematic representation of the experimental procedure involved in the temperature quench method for constructing a phase diagram for anti-A1 antibody formulated at 70 mg/ml in 10 mM Tris pH 8.

FIG. 4 is a phase diagram for anti-A1 antibody formulated at 70 mg/ml in 10 mM Tris pH 8 that was constructed using the temperature quench method. The region that is considered “beyond or outside” the phase diagram is shaded with slanted lines. The region considered to be “inside or under” the phase diagram is filled in.

FIG. 5 is a phase diagram for anti-Al antibody that was constructed using the cloud-point method. The region that is considered “beyond or outside” the phase diagram is shaded with slanted lines. The region considered to be “inside or under” the phase diagram is filled in.

FIG. 6 provides a correlation of a phase diagram with the appearance of four anti-A1 antibody formulations formulated at different concentrations in 10 mM Tris pH 8 at a fixed temperature of 10° C. The lower and upper phase boundary concentrations at 10° C. are indicated on the diagram by asterisks. This figure shows that protein formulations formulated using concentrations “outside or beyond” the phase diagram are clear, while those formulations having concentrations that fall “within or inside” the phase diagram are opalescent.

FIG. 7 provides a comparison of the concentration of excipients in the upper and lower phases of an anti-A1 antibody formulation formulated at 59 mg/ml in 10 mM Tris pH 8.0 that has undergone liquid-liquid phase separation. The “post-dialysis” sample corresponds to the protein formulation prior to liquid-liquid phase separation.

FIG. 8 provides a comparison of the size (SEC-HPLC) and charge (CEX-HPLC) of proteins in the upper and lower phase of an anti-A1 antibody formulation formulated at 59 mg/ml in 10 mM Tris pH 8.0 that has undergone liquid-liquid phase separation.

FIG. 9 provides a comparison of the secondary structure of anti-A1 in the upper and lower phases of an anti-A1 antibody formulation formulated at 59 mg/ml in 10 mM Tris pH 8.0 that has undergone liquid-liquid phase separation, using FTIR amide I spectrum.

FIG. 10 provides a comparison of the tertiary structure of the proteins in the upper and lower phases of an anti-A1 antibody formulation formulated at 59 mg/ml in 10 mM Tris pH 8.0 that has undergone liquid-liquid phase separation, using fluorescence with a 45° angle cuvette.

### DETAILED DESCRIPTION OF THE INVENTION

There is a growing demand to achieve high protein concentrations for protein-based therapeutics. However, because of the phenomenon of liquid-liquid phase separation in certain protein solutions formulated at high protein concentrations, opalescence (i.e., cloudiness of the protein solution) is a major problem for protein formulators. Methods disclosed herein provide novel processes for formulating proteins at high concentrations by reducing or preventing liquid-liquid phase separation at a desired temperature. Thus, these methods yield protein solutions having reduced opalescence. The methods described herein use liquid-liquid phase separation and/or phase diagrams to determine concentrations that are suitable for formulating a protein in a buffer/solution of interest, for use at a desired temperature such that the resulting high protein concentration solutions have reduced liquid-liquid phase separation and are visibly clear. The methods described herein also utilize liquid-liquid phase separation and phase separation curves to determine temperatures that are suitable for formulating a protein in a buffer/solution of interest, for use at a fixed concentration such that the resulting protein solutions have reduced liquid-liquid phase separation and are visibly clear. Furthermore, the invention provides methods for concentrating protein solutions, and purifying proteins.

Relationship Between Solubility and Opalescence

The solubility of a protein in a protein solution is related to the opalescence of the protein solution. By “opalescence” is meant a detectable cloudiness or turbidity of a protein solution. It has been found by the inventors that an inverse relationship exists between solubility and opalescence, especially at high protein concentrations. More specifically, decreased solubility of a protein in a protein solution correlates with increased opalescence of the protein solution at high concentrations of the protein in the protein solution. This information is useful to a formulator in determining how to formulate a protein at high concentration. By “high concentration” is meant a protein concentration greater than about 50 mg/ml (e.g., 50 mg/ml, 75 mg/ml, 100 mg/ml, 200 mg/ml, 300 mg/ml, 400 mg/ml, 500 mg/ml, and so on). As used throughout this application, the term “about” means a numeral value that is ±20% around the cited value. If a particular protein solution is cloudy, the formulator can then find alternative methods to formulate the protein to reduce or prevent cloudiness of the protein solution. For example, the solubility of a protein of interest at high concentrations may be tested in several buffers/solutions to determine the solubility of the protein in that buffer. If the solubility is low in a particular buffer/solution, it is likely that the protein solution will be opalescent. Accordingly, the formulator can choose a different buffer/solution in which the protein at high concentrations is more soluble, thereby enabling the formulator to achieve a clear protein formulation.

Methods of determining the solubility of a protein in a protein solution are well known in the art. U.S. Provisional Appl. No. 60/801,862, incorporated by reference herein, describes a novel method of predicting the relative solubility of a protein using polyethylene glycol precipitation.

Opalescence can generally be determined by simple visualization by eye (i.e., if a solution is cloudy or turbid), right angle light scattering, or fluorescence. In some cases, however, opalescence is not detectable to the human eye. In these cases, and in cases where a quantitative read out is desired, opalescence can be determined using more sensitive methods such as spectrophotometry e.g., automated spectrophotometry by using a visible light spectrophotometer (with measurements in the 400-600 nm range, e.g., 500 nm), or equivalent means for detecting light absorbance of the samples. Other methods for assaying opalescence include photo-electric turbidometery (e.g., automated turbidometry).

Opalescence and Liquid-Liquid Phase Separation

Liquid-liquid phase separation can lead to opalescence of a protein solution. By “liquid-liquid phase separation,” also known as binary-liquid phase separation and coacervation, is meant a phenomenon by which a protein solution separates into two coexisting liquid phases of unequal protein concentrations at a temperature less than the critical temperature for phase separation. Liquid-liquid phase separation is usually observed in protein solutions having a high concentration of protein; such protein solutions are opalescent.

Several factors play a role in liquid-liquid phase separation. These include, but are not limited to, the temperature of the protein solution, the concentration of the protein(s) in the protein solution, pH of the solution or buffer, the ionic strength of the solution or buffer, and the protein's self-interaction and solubility properties. With respect to temperature, liquid-liquid phase separation is observed when the temperature of the solution is below the critical temperature for phase separation. By “critical temperature for phase separation” is meant the temperature at which a protein solution has the potential to undergo liquid-liquid phase separation. With respect to concentration, high concentration protein solutions are also more likely to exhibit phase separation. With respect to pH, if the pH of a protein solution formulated at a high protein concentration is close to the protein's isoelectric point (pI), liquid-liquid phase separation is likely to occur in the protein solution. Finally, increased ionic strength in a protein solution formulated at a high protein concentration favors liquid-liquid phase separation.

Liquid-liquid phase separation can be facilitated by mixing a high concentration protein solution and allowing the protein solution to settle. Gravity separation leads to the separation of an upper liquid phase and a lower liquid phase separated by a narrow meniscus. In addition to gravity separation, an alternative method to facilitate separation of the two phases is centrifugation (e.g., at 4,000×g). For the same protein solution, sedimentation velocity is greater at lower temperature due to a larger difference in density between the two phases. The lower phase can be separated from the upper phase by any means known to the ordinarily skilled artisan, e.g., by using a Pasteur pipet.

The upper phase and lower phase of a protein solution that has undergone liquid-liquid phase separation have different protein concentrations. The lower phase has a higher protein concentration than the upper phase. The concentration of protein in the upper and lower phase can be determined by any method known in the art, including, but not limited to, measurement of the protein's intrinsic UV absorbance (at 280 nm), the Lowry assay, the Smith copper/bicinchoninic assay, and the Bradford dye assay.

To determine if an unknown protein when formulated at a specific high concentration in a buffer/solution of choice at a desired temperature will undergo liquid-liquid phase separation, the protein is formulated at the specific concentration in the desired buffer/solution, mixed and incubated at the desired temperature. If the mixed protein solution is cloudy, it is a first indication that liquid-liquid phase separation is likely to occur. Of course, after gravity separation or centrifugation, it will be readily apparent if the protein solution separates into upper and lower liquid phases.

Constructing a Phase Diagram

If a protein solution undergoes liquid-liquid phase separation in a desired buffer/solution at a desired temperature, it is necessary to find methods to avoid this phenomenon. This is especially important for pharmaceutical formulations as liquid-liquid phase separation creates non-homogenous solutions that are also cloudy in appearance. The methods described herein provide a solution for this problem. In some embodiments, these methods relate to the use of a phase diagram.

In the context of the instant application, a phase diagram, also known as a phase separation curve or a coexistence curve, is a map that indicates the phase behavior (i.e., if a protein solution exists in one phase or two phases) as a function of concentration of a protein in a protein solution, and the temperature of the protein solution. The phase diagram specifies the two concentrations of the liquid phases coexisting in equilibrium at a given temperature. Phase diagrams are usually arrived at by one of two methods: (i) the temperature quench method, or (ii) the cloud-point temperature method.

Temperature Quench Method

In a non-limiting example of this method for constructing a phase diagram, several samples of a protein solution containing the same high concentration of a protein (e.g., about 50-100 mg/ml) are cooled from a temperature at which the protein solution does not exhibit liquid-liquid phase separation to lower temperatures. The temperature at which the protein solution does not exhibit liquid-liquid phase separation can be determined empirically. Usually, the higher the temperature, the lower the chance of a protein solution exhibiting liquid-liquid phase separation. Typically, one can start at room temperature, and if phase separation is observed for the protein solution of interest at that temperature, one can move to higher temperatures. Once the temperature at which phase separation is not found to occur is identified, all the protein solutions are brought to that temperature from their existing temperatures. These samples are then cooled to several different lower temperatures at which liquid-liquid phase separation occurs. The samples can be incubated in a temperature controlled chamber for about 30 minutes and then visually checked for whether the solution is cloudy or clear. The cloudy samples are then allowed to equilibrate into upper and lower phases by gravity separation or centrifugation. Aliquots of the samples from the upper and lower phases of each protein solution are taken and the protein concentration in these samples is measured. A phase diagram is constructed by plotting the concentration of the upper and lower phases of each of the protein solutions at the particular temperature of the protein solutions at which the solution exhibits liquid-liquid phase separation. Typically, the temperature is plotted on the y-axis and the protein concentration on the x-axis. A non-limiting illustration of this method for constructing a phase diagram is provided in Example 3.

Cloud-Point Method

In a non-limiting example of this method, one or more protein solutions of known protein concentration and at a temperature at which the protein solutions are not opalescent are placed in a light-scattering spectrophotometer and the temperature is then slowly lowered. The temperature at which the protein solution is not opalescent can be determined empirically. For example, one can start at room temperature and move to higher temperatures, as needed, until one obtains a solution that is not cloudy at a given concentration. The intensity of light scattering for the protein solutions is monitored at each temperature. The onset of phase separation in the protein solutions is detected by the disappearance of the transmitted beam due to extensive multiple scattering of the incident beam. This corresponds to the solution turning cloudy. The temperature at which phase separation commences, and thus opalescence sets in is the cloud point temperature, T_{Cloud}. The T_{Cloud} of each protein solution is then plotted against the concentration of that protein solution to obtain a phase diagram based on cloud-point temperature. Typically, the temperature is plotted on the y-axis and the concentration on the x-axis. A non-limiting illustration of this method is provided in Example 4.

Interpreting a Phase Diagram

The phase diagram for liquid-liquid phase separation of a high concentration protein solution resembles an inverted parabola. The ascending limb and the descending limb of the curve come together at the upper most point on the curve, which defines the critical temperature (T_{c}). The critical temperature represents the temperature at which phase separation can begin to occur so long as the concentration of the protein in a protein solution is in the appropriate range. In the region outside the phase separation curve (i.e., outside the inverted parabola) the protein solution is homogenous and in a single phase. In the region under the curve, liquid-liquid phase separation occurs, and sedimentation of the high density phase leads to macroscopic separation into two transparent layers with high protein concentration in the bottom liquid phase and low protein concentration in the upper liquid phase.

Methods for Determining a Concentration for Formulating a Protein Solution Having Reduced Opalescence

The application relates in part to methods for determining a concentration at which to formulate a protein in a buffer/solution wherein the resulting protein solution does not exhibit, or exhibits reduced liquid-liquid phase separation at a desired temperature(s). The application also relates in part to methods for determining a concentration at which to formulate a protein in a buffer/solution so that the protein solution is substantially clear (i.e., has reduced opalescence) at a desired temperature(s). These methods are useful in determining a new concentration at which to reformulate a protein in a buffer/solution, when the originally formulated protein solution exhibits liquid-liquid phase separation at a desired temperature(s).

In general, these methods involve determining that a concentration of a protein for formulation in a buffer/solution, is to be a concentration that is outside the phase diagram for the protein solution at a desired temperature(s). If the protein solution is formulated using a protein concentration that is outside and to the left (lower concentration protein solution), or outside and to the right of the curve (higher concentration protein solution) at a desired temperature, the protein solution will not exhibit liquid-liquid phase separation or opalescence at those concentrations at the desired temperature. By “desired temperature” is meant the temperature at which the protein is to be used, manipulated or stored. Non-limiting examples of a desired temperature include the temperature at which a protein solution is to be manufactured, processed, stored, or administered to a subject.

Typically when formulators formulate protein solutions of increasing protein concentration, they do not attempt to increase the concentration of a protein solution beyond a concentration at which opalescence or liquid-liquid phase separation sets in. Here, the inventors have surprisingly and unexpectedly found that as long as the protein concentration is increased beyond or outside the phase diagram at a desired temperature, the cloudiness or liquid-liquid phase separation can be reduced or even disappear. This is an extremely important finding for formulators trying to formulate high concentration protein (e.g., monoclonal antibody) formulations for use in therapy.

It is not necessary, however, that a phase diagram be prepared for the protein solution to practice the methods described herein, if it is known a priori that a protein solution is needed at a desired temperature. This method can be used, for example, when a formulator formulates a protein in a buffer/solution at a certain concentration and finds that the formulated solution undergoes liquid-liquid phase separation, or is opalescent, at the desired temperature. In this instance, the formulator can determine the concentrations of the lower and/or the upper phase of the protein solution that has undergone liquid-liquid phase separation. To obtain a high concentration protein solution that does not exhibit phase separation at the desired temperature, one would choose a concentration that is greater than the concentration of the lower phase (i.e., the phase having the higher protein concentration). To obtain a low concentration protein solution that does not exhibit liquid-liquid phase separation at the desired temperature, one would choose a concentration that is lower than the concentration of the upper phase. If one is only interested in formulating a high concentration protein solution, it is sufficient that the concentration of the lower layer of the protein solution that has undergone liquid-liquid phase separation be measured. If the protein is formulated at a concentration higher than the concentration of the lower layer for use at the temperature at which the phase separation occurred, the resulting protein formulation exhibits reduced or no liquid-liquid phase separation and will be substantially clear at the desired temperature. In certain embodiments, the protein concentration is chosen to be about 0.5% to about 5%, about 5% to about 10%, about 10% to about 20%, about 20% to about 30%, about 30% to about 40%, about 5% to about 20%, about 5% to about 30%, or about 5% to about 40% higher than the concentration of the protein in the lower phase.

In another aspect, the methods relate to determining a concentration at which to formulate a protein in a protein solution by constructing a phase diagram. The phase diagram can be prepared by any method including, but not limited to, the temperature quench method and the cloud-point method.

If the temperature quench method is used to construct a phase diagram, at least two protein solutions having the same protein concentration and that undergo phase separation at two different temperatures are used. In some embodiments, at least three protein solutions having the same protein concentration and that undergo phase separation at three different temperatures are used. In other embodiments, at least four protein solutions having the same protein concentration and that undergo phase separation at four different temperatures are used. In yet other embodiments, at least five protein solutions having the same protein concentration and that undergo phase separation at five different temperatures are used. Any variation of the temperature quench method described above can be used to construct the phase diagram for the protein solutions.

If the cloud-point method is used to construct a phase diagram, at least two protein solutions having different protein concentrations of the same protein are used. In some embodiments, at least three protein solutions having different protein concentrations of the same protein are used. In other embodiments, at least four protein solutions having different protein concentrations of the same protein are used. In yet other embodiments, at least five protein solutions having different protein concentrations of the same protein are used.

Regardless of the method used, the phase diagram will permit the formulator to choose a protein concentration at which a protein solution will have a reduced likelihood of exhibiting liquid-liquid phase separation at a desired temperature or range of temperatures. Specifically, the formulator will look at the temperature or range of temperatures at which the formulation is to be used, stored, processed, and/or manufactured and based on that temperature choose a concentration that is outside the phase diagram for that temperature. In certain embodiments, the protein concentration is chosen to be about 0.5% to about 5%, about 5% to about 10%, about 10% to about 20%, about 20% to about 30%, about 30% to about 40%, about 5% to about 20%, about 5% to about 30%, or about 5% to about 40% higher than the highest concentration of the protein on the phase separation curve corresponding to the desired temperature (i.e., the upper phase boundary concentration). By choosing a concentration that is outside the phase diagram for several different temperatures of interest for a given protein solution, one can formulate a protein in a buffer/solution that does not exhibit liquid-liquid phase separation for that range of temperatures. This is particularly useful where a given protein solution needs to be stored, used, processed, or manufactured at different temperatures.

Methods of Formulating Protein Solutions Having Reduced Opalescence

The methods described herein are useful for formulating proteins at high concentrations wherein the resulting protein solution exhibits reduced or no opalescence. In a specific embodiment, the methods are useful in instances where a protein must be formulated in a specific buffer/solution and wherein the protein exhibits liquid-liquid phase separation in that buffer/solution at a specific concentration and at the desired temperature. In another specific embodiment, the methods of the present application are useful where it is necessary to reformulate a protein from one buffer/solution to another buffer/solution, and wherein the protein when formulated in the new buffer/solution exhibits liquid-liquid phase separation. In such cases, using the concentrations identified from a phase diagram for the protein solutions that show liquid-liquid phase separation, one can formulate a protein in a buffer/solution such that the protein solution does not exhibit liquid-liquid phase separation at a desired temperature.

For formulating proteins in buffers/solutions at high concentrations for use at a fixed temperature, it is sufficient that the protein concentration be outside or beyond the phase separation curve for that protein solution at the desired temperature. The proteins may be formulated at any concentration higher than the highest concentration on the curve for the desired temperature. Generally, the proteins are formulated at a concentration that is between about 0.5% to about 5%, about 5% to about 10%, about 10% to about 20%, about 20% to about 30%, about 30% to about 40%, about 5% to about 20%, about 5% to about 30%, or about 5% to about 40% higher than the highest concentration of the protein on the phase separation curve corresponding to the desired temperature (i.e., the upper phase boundary concentration).

If the protein solution begins to become viscous or shows signs of aggregation, as the concentration of the protein in a protein formulation increases, any methods for reducing viscosity and/or reducing aggregation can be employed to reduce the viscosity and/or aggregation of the protein solution (e.g., U.S. Provisional Appl. Nos. 60/752,660 and 60/784,130, both of which are incorporated by reference herein). In some embodiments, to reduce viscosity of a high concentration protein solution, one can add between about 0.5 mM to 25 mM of calcium chloride or magnesium chloride (e.g., about 0.5 mM to about 5 mM, about 2 mM to about 10 mM, about 5 mM to about 10 mM, about 10 mM to about 15 mM, about 15 mM to about 20 mM, about 13 mM to about 25 mM) to the protein formulation. In other embodiments, to reduce aggregation of a high concentration protein solution, one can add between about 1 mM to about 145 mM methionine (e.g., about 1 mM to about 5 mM, about 5 mM to about 10 mM, about 10 mM to about 20 mM, about 20 mM to about 50 mM, about 50 mM to about 100 mM, about 100 mM to about 140 mM). In additional embodiments, both calcium chloride/magnesium chloride and methionine may be added in the ranges defined above to a high concentration protein formulation.

To reformulate a protein at a fixed concentration in a protein solution, so as to reduce or prevent liquid-liquid phase separation in the protein solution, one can again use a phase diagram. In this case, one would increase the temperature to be above the highest temperature on the phase diagram corresponding to the desired concentration. In certain embodiments, the temperature is increased by about 1° C., about 2° C., about 3° C., about 4° C., about 5° C., about 6° C., about 7° C., about 8° C., about 9° C., about 10° C., between about 0.5° C. to about 5° C., between about 0.5° C. to about 10° C., or between about 5° C. to about 10° C., above the highest temperature on the phase diagram corresponding to the desired concentration.

The invention also relates to methods of obtaining a protein solution that does not exhibit or shows reduced liquid-liquid phase separation from a protein solution that exhibits liquid-liquid phase separation. The method generally involves facilitating liquid-liquid phase separation of the protein solution that exhibits liquid-liquid phase separation and separating the lower phase of the protein solution. Liquid-liquid phase separation can be facilitated by, for example, gravity separation or centrifugation. The lower phase can be separated from the upper phase by any method known in the art, including using a Pasteur pipet. The lower phase of the protein solution is a protein solution that does not exhibit or shows reduced liquid-liquid phase separation compared to the original protein solution.

Methods of Concentrating Proteins

The application also relates to methods of concentrating proteins. In one embodiment, a method of obtaining a higher concentration protein solution from a lower concentration protein solution that exhibits liquid-liquid phase separation is provided. The method involves facilitating liquid-liquid phase separation of the original protein solution and separating the lower phase from the upper phase. The lower phase is a protein solution that has a higher concentration of the protein than the original lower concentration protein solution.

This method can be adapted so as to concentrate a protein from a lower concentration protein solution into a completely new buffer/solution. The method comprises the steps outlined above, but further includes a buffer exchange step to transfer the protein to the new buffer/solution. Methods of conducting buffer exchange can be performed using any methods known in the art, including, but not limited to, membrane-based methods such as dialysis, ultrafiltration, and diafiltration.

If, upon concentration, the protein solution begins to become viscous or shows signs of aggregation, any methods for reducing viscosity and/or reducing aggregation can be employed to reduce the viscosity and/or aggregation of the protein solution (e.g., U.S. Provisional Appl. Nos. 60/752,660 and 60/784,130). In some embodiments, to reduce viscosity of a high concentration protein solution, one can add between about 0.5 mM to 25 mM of calcium chloride or magnesium chloride (e.g., about 0.5 mM to about 5 mM, about 2 mM to about 10 mM, about 5 mM to about 10 mM, about 10 mM to about 15 mM, about 15 mM to about 20 mM, about 13 mM to about 25 mM) to the protein formulation. In other embodiments, to reduce aggregation of a high concentration protein solution, one can add between about 1 mM to about 145 mM methionine (e.g., about 1 mM to about 5 mM, about 5 mM to about 10 mM, about 10 mM to about 20 mM, about 20 mM to about 50 mM, about 50 mM to about 100 mM, about 100 mM to about 140 mM). In additional embodiments, both calcium chloride/magnesium chloride and methionine may be added in the ranges defined above to a high concentration protein formulation. Methods of measuring viscosity and aggregation are well known to one of skill in the art.

Methods of Purifying Proteins

Liquid-liquid phase separation can also be utilized to purify a desired protein from a mixture of other proteins. Such purification is based on the careful choice of phase separation conditions based on the protein of interest. If the chosen phase separation condition (e.g., pH, ionic strength) is unique to the protein of interest, but not the rest of the protein mixture from which the protein is to be purified, then only the protein of interest will phase separate under the chosen phase separation conditions, and the protein rich bottom layer can be collected as a pure protein solution.

Liquid-liquid phase separation is particularly useful for purifying antibodies or antigen-binding fragments thereof. This is because unlike most proteins that have an isoelectric point (pI) of less than 7 (often much lower than 7), most antibodies have a pI of between about 8 and 9. Because of these high pI's, antibodies can be phase separated from other proteins using a buffer having a pH that is close to their pI. In this way, the antibodies will be found at high concentrations in the lower layer while the remaining proteins will be in the upper layer. For example, in the process of purifying anti-A1 antibody, the protein solution containing anti-A1 and other proteins from cell debris and culture media can be buffer exchanged into a pH 8.0 buffer, which is the optimal phase separation condition for anti-A1 antibody. Under these conditions, anti-A1 will settle into two phases and the bottom high-concentration layer can be collected as purified anti-A1 antibody.

Of course, that the above procedures for purification can also be used for proteins, other than antibodies, that have high isoelectric points.

If a buffer condition can cause more than one protein to undergo phase separation in a buffer having a pH of about 8-10, then it is possible to simultaneously purify two or more proteins from a mixture of other proteins. Even if only a single protein is desired, this procedure will provide a protein that is more purified than the original protein mixture. As noted above, liquid-liquid phase separation can be achieved by gravity or facilitated by centrifugation.

Proteins

The methods described herein are generally useful for any protein. “Protein” as used herein includes proteins, peptides, protein fragments, conjugated proteins, and polypeptides that contain non-naturally-occurring amino acids. Proteins can be obtained from any source, for example, secreted recombinant proteins, proteins isolated from natural sources, non-secreted recombinant proteins, or synthetic proteins. In certain embodiments, the proteins include, but are not limited to, antibodies, antigen-binding antibody fragments, ligand-binding molecules, soluble receptors, ligands, coagulation factors, signaling proteins, and transcription factors.

The term “antibody,” as used herein, includes polyclonal antibodies, monoclonal antibodies, antibody compositions with polyepitope specificities, bispecific antibodies, diabodies, or other purified preparations of antibodies and recombinant antibodies. The antibodies may be whole antibodies, e.g., of any isotype (IgG, IgA, IgE, IgM, etc.), or fragments thereof, which bind the protein of interest. Antibodies can be fragmented using conventional techniques and the fragments screened for binding to the antigen of interest. Preferably, an antibody fragment comprises the antigen binding and/or the variable region of an intact antibody. Thus, the term antibody fragment includes segments of proteolytically cleaved or recombinantly-prepared portions of an antibody molecule that are capable of selectively binding a certain protein. Non-limiting examples of such proteolytic and/or recombinant fragments include Fab, F(ab′)₂, Fab′, Fv, and single chain antibodies (scFv) containing a V[L] and/or V[H] domain joined by a peptide linker. The scFv's may be covalently or noncovalently linked to form antibodies having two or more binding sites.

In some embodiments the antibody is a humanized monoclonal antibody. The term “humanized monoclonal antibody,” as used herein, is a monoclonal antibody from a non-human source (recipient) that has been altered to contain at least one or more of the amino acid residues found in the equivalent human monoclonal antibody (donor). A “fully humanized monoclonal antibody” is a monoclonal antibody that has been altered to contain all of the amino acid residues found in the antigen-binding region of the equivalent human monoclonal antibody. Humanized antibodies may also comprise residues which are not found either in the recipient antibody or the donor antibody. These modifications may be made to further refine and optimize antibody functionality. The humanized antibody may also optionally comprise at least a portion of a human immunoglobulin constant region (Fc).

In certain embodiments, the protein to be formulated, concentrated and/or purified does not include lyzozyme, γ-crystallins, β-lactoglobulin, thaumatin, concanavalin A, and catalase.

Protein concentrations suitable for use in the methods disclosed herein include, but are not limited to, 5 mg/mL to about 50 mg/mL, about 50 mg/mL to about 100 mg/mL, about 100 mg/mL to about 200 mg/mL, about 200 mg/mL to about 300 mg/mL, and about 300 mg/mL to about 500 mg/mL.

### EXAMPLES

The invention is further illustrated by the following examples. The examples are provided for illustrative purposes only. They are not to be construed as limiting the scope or content of the invention in any way.

### Example 1

### Determination of the Relationship Between Solubility and Opalescence

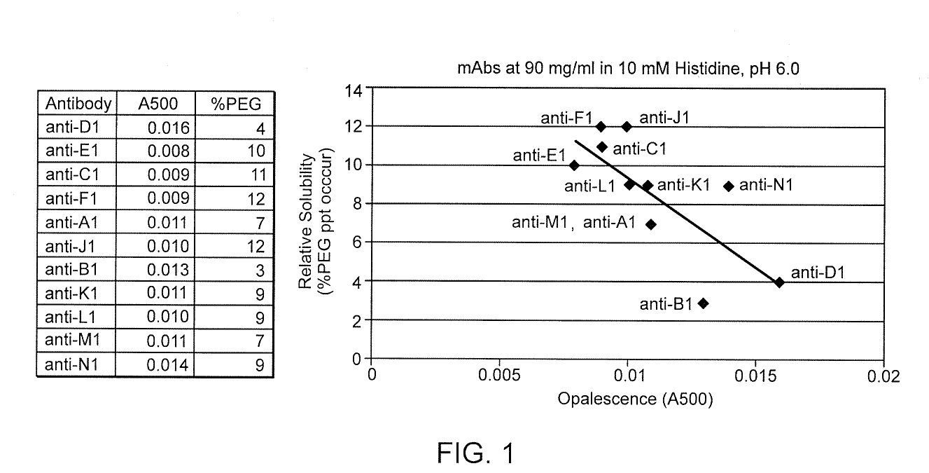

To test if a relationship exists between solubility and opalescence, 11 different monoclonal antibodies formulated at 90 mg/ml in 10 mM Histidine pH 6.0 were studied. These antibody solutions were tested for solubility and opalescence. Solubility was assessed by PEG precipitation as described in U.S. Provisional Appl. No. 60/801,862. Opalescence was measured by measuring the absorbance of the antibody solution at 500 nm. The data from these studies which are shown in FIG. 1 indicate that the lower the solubility of the protein, the greater the opalescence of the protein solution.

Thus, there is an inverse relationship between solubility and opalescence at high protein concentrations.

### Example 2

### Liquid-Liquid Phase Separation

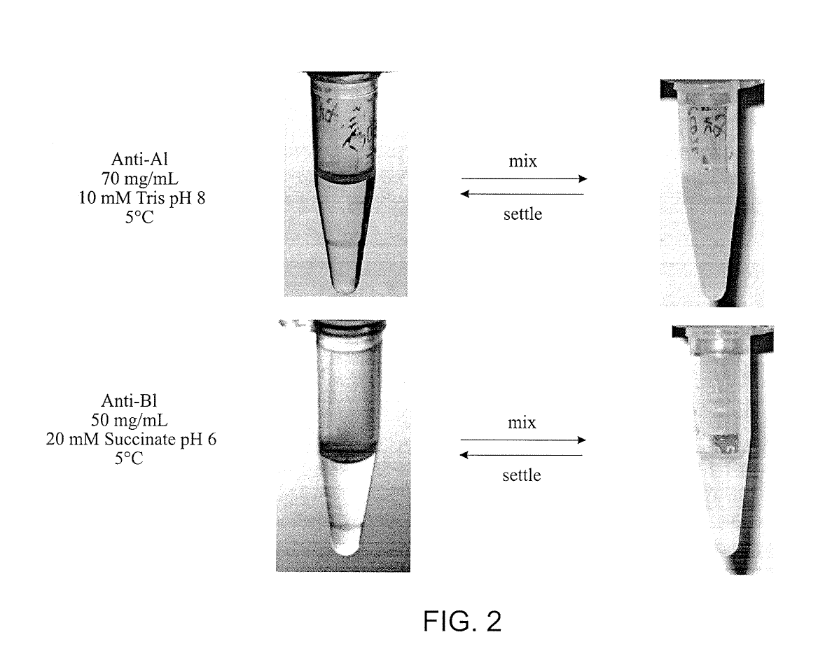

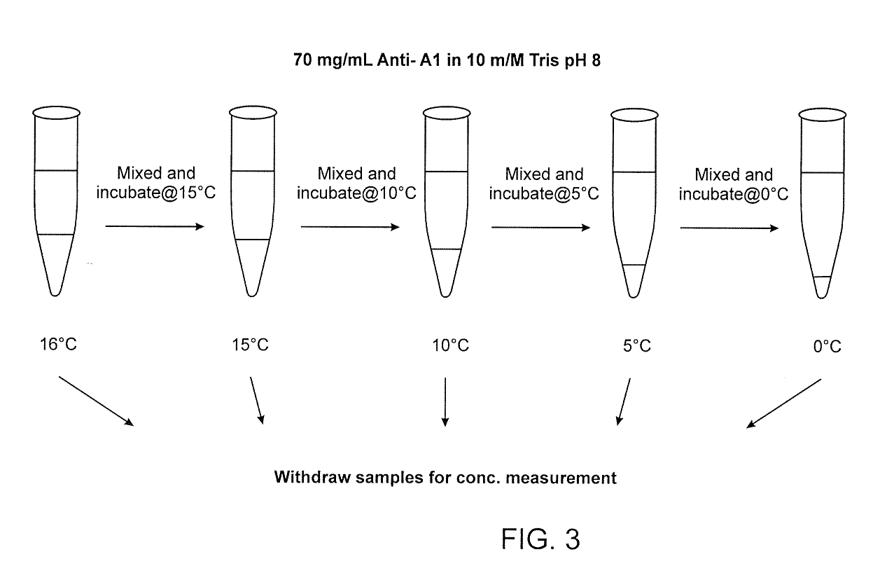

Anti-A1 monoclonal antibody was formulated at 70 mg/ml in 10 mM Tris pH 8.0 at 5° C. The pH of this protein solution is close to the pI of the antibody. When the protein solution was mixed it appeared cloudy; however, when it was allowed to settle, liquid-liquid phase separation occurred resulting in an upper and lower phase (see, FIG. 2, upper panel).

Anti-B1 monoclonal antibody was formulated at 50 mg/ml in 20 mM succinate pH 6.0 at 5° C. When this solution was mixed, the solution became cloudy; however, when it was allowed to settle, liquid-liquid phase separation occurred, resulting in an upper and lower phase (see, FIG. 2, bottom panel).

A similar liquid-liquid phase separation phenomenon was observed when an anti-C1 antibody formulated at 90 mg/ml in 10 mM Tris pH 9.0 at 5° C. was mixed and allowed to settle (data not shown).

Thus, liquid-liquid phase separation is observed in several proteins at high concentration. The pH, ionic strength and solubility properties may contribute to the observed phase separation. As seen in this example, protein solutions that exhibit liquid-liquid phase separation are opalescent.

### Example 3

### Constructing a Phase Diagram for an anti-A1 Protein Solution by the Temperature Quench Method

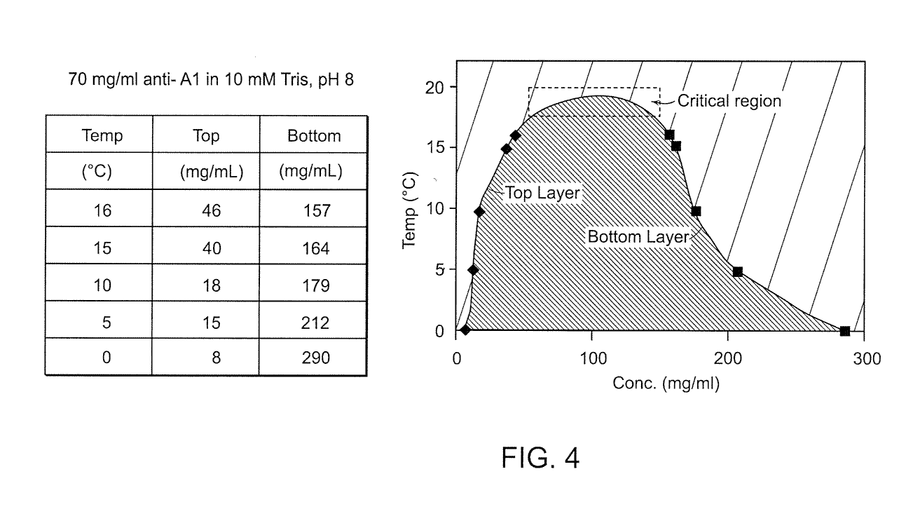

Five samples of anti-A1 monoclonal antibody, each of which was formulated at 70 mg/ml in 10 mM Tris pH 8.0, were cooled from room temperature to 16° C., 15° C., 10° C., 5° C., and 0° C., respectively, and allowed to undergo a macroscopic gravity-driven separation until two clear liquid layers were achieved by gravity sedimentation. Aliquots from the upper and lower layers were isolated from each sample, and the concentration of each layer was measured UV-vis spectroscopy (280 nm). The concentration of the upper and lower layers was plotted against the temperature of the protein solution to obtain a phase diagram (see, FIG. 4).

Anti-A1 protein solution underwent a liquid-liquid phase separation when cooled below a critical temperature in this buffer. In the region under the curve, liquid-liquid phase separation occurs, and the sedimentation of the high-density phase leads to macroscopic separation into two transparent layers, with high protein concentration phase at the bottom and the low protein concentration phase on the top. The two liquid phases with different protein concentrations coexist in equilibrium at a given temperature.

### Example 4

### Constructing a Phase Diagram for an Anti-A1 Protein Solution by the Cloud-Point Method

Four samples of anti-A1 antibody formulated at 200 mg/ml, 50 mg/ml, 30 mg/ml and 10 mg/ml in 10 mM Tris pH 8.0 were prepared. These four solutions were placed in a light-scattering spectrophotometer at 30° C. At this temperature, all of these solutions were clear. When these solutions were slowly cooled, phase separation commenced in each solution at a particular temperature (T_{cloud}). This phenomenon was marked by the disappearance of the transmitted beam due to extensive multiple scattering of the incident beam. Below this T_{cloud} temperature, the solution turned opaque. For each of the solutions, the cloud-point temperature was plotted against the concentration of the protein solution to arrive at a phase diagram for the protein solution.

It is noteworthy that both the temperature quench method (Example 3) and the cloud-point method of constructing phase diagrams appear to give substantially similar results.

### Example 5

### Predicting Opalescence Using a Phase Diagram

To determine whether a phase diagram can be used to determine whether a given protein solution is opalescent (i.e., cloudy) or clear, four different anti-A1 protein solution samples were prepared at different concentrations in 10 mM Tris pH 8.0 (i.e., the same protein and buffer used in the experiments to create the phase diagram) that fell in different regions of the phase diagram (i.e., inside or outside the phase separation curve).

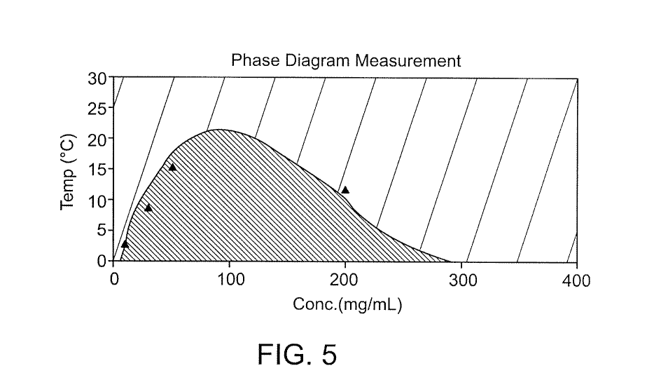

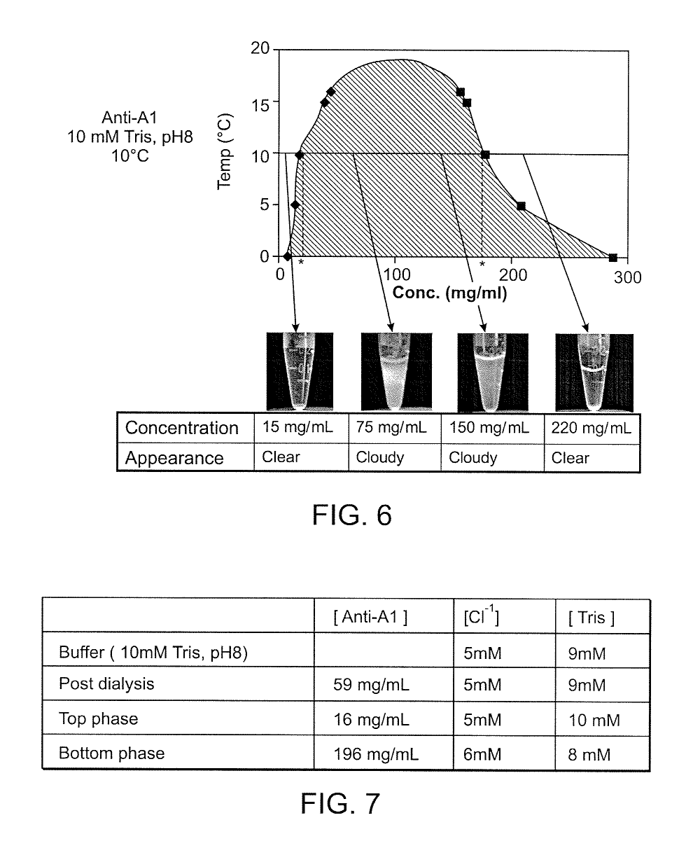

As shown in FIG. 5, the phase diagram correlates well with the occurrence of opalescence. The protein solution is clear and remains in a single phase outside the phase separation curve, but turns cloudy and separates into two layers upon gravity separation under the phase separation curve. The phase diagram demonstrates that at a fixed temperature of 10° C., anti-A1 solution starts to turn opaque as the concentration reaches above 18 mg/ml, but unexpectedly clears up as the concentration continues to increase above 180 mg/ml.

Therefore, for a protein solution that has a tendency towards opalescence due to liquid-liquid phase separation, a clear solution at high protein concentration at a certain temperature is achievable as long as the concentration is beyond the phase separation curve.

### Example 6

### Analysis of the Properties of the Upper and Lower Phases of a Liquid-Liquid Phase Separation

Anti-A1 formulated at 59 mg/ml in 10 mM Tris pH 8.0 was allowed to undergo liquid-liquid phase separation, and aliquots from the upper and lower phases were isolated and used to analyze the following properties: (i) buffer components, (ii) high molecular weight species, (iii) acidic species, (iv) secondary structure, and (v) the tertiary structure of the proteins in both phases.

An analysis of the buffer components indicated that the concentration of Tris and the chloride ion in both phases were substantially identical (see, FIG. 7).

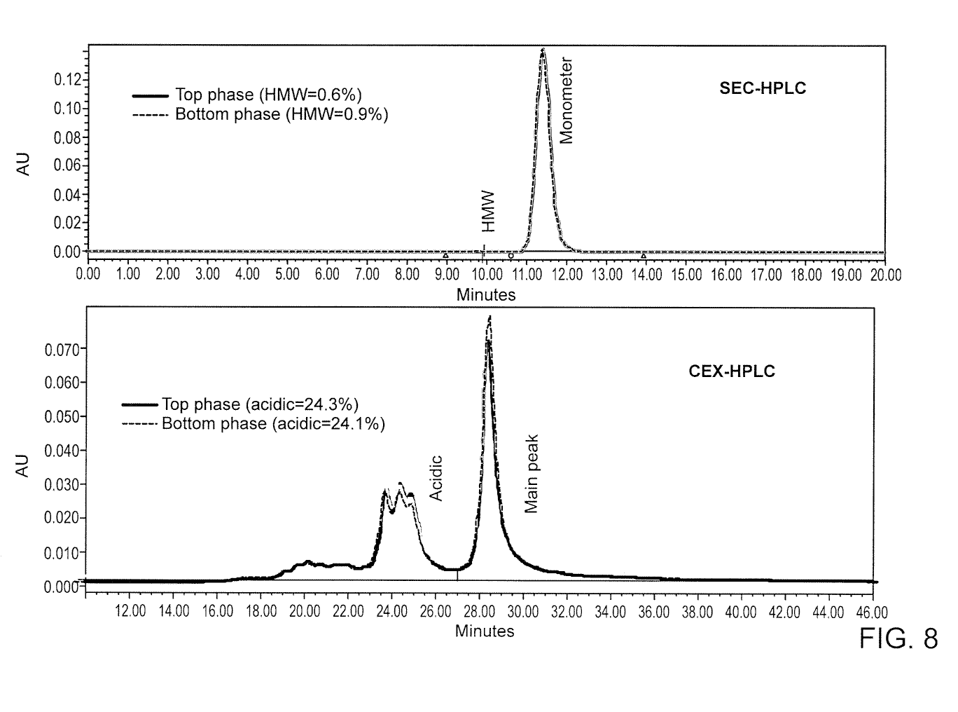

An analysis of the high molecular weight species by size exclusion-high performance liquid chromatography indicated no significant differences in the upper and lower phase (see, FIG. 8, upper panel).

Similarly, an analysis of the acidic species by cation exchange-high performance liquid chromatography in both phases, showed no significant differences (see, FIG. 8, lower panel).

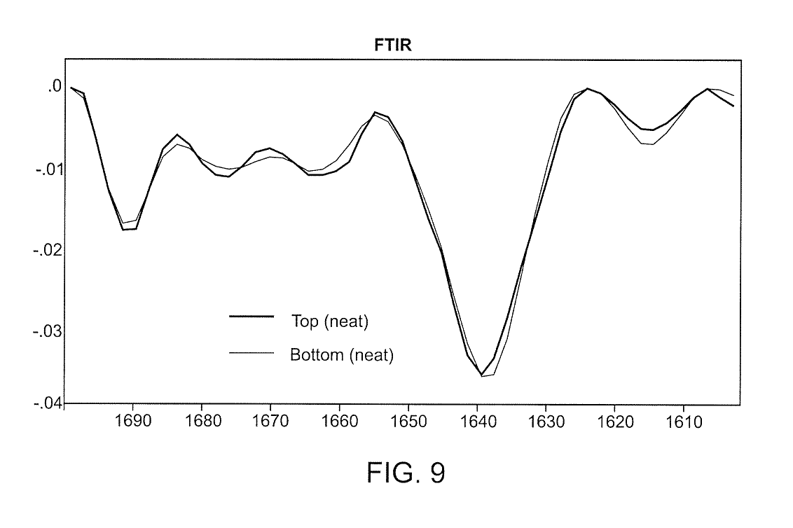

The secondary structure of anti-Al from both phases as characterized by FTIR amide I spectrum was substantially similar (see, FIG. 9).

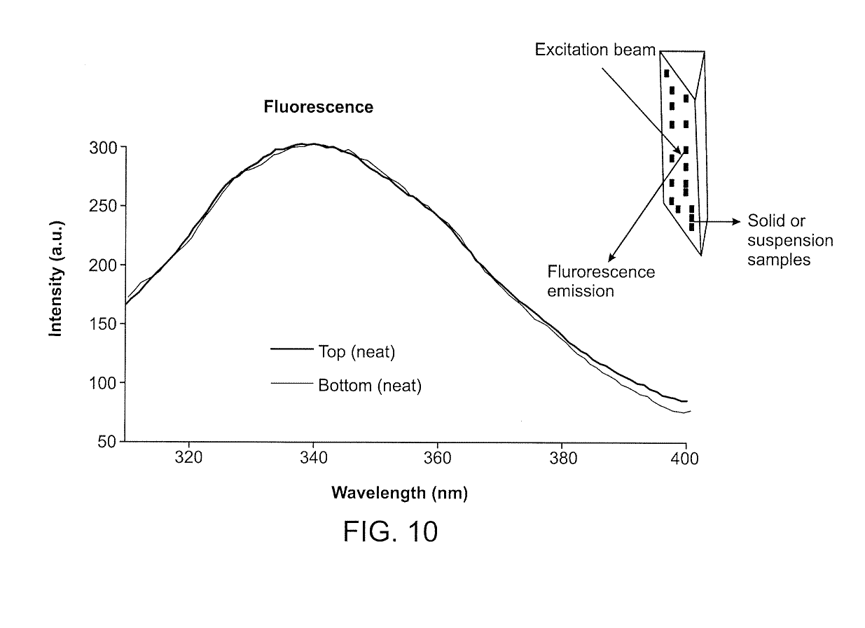

Finally, the tertiary structure of anti-A1 from both phases as characterized by fluorescence using a 45° angle cuvette also showed no significant differences (see, FIG. 10).

Taken together, these data suggest that the upper and lower phase of a protein solution that undergoes liquid-liquid phase separation are substantially similar. Thus, differences in secondary or tertiary structure of the protein, physicochemical properties or excipient content are likely not the underlying cause for liquid-liquid phase separation.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method for determining a concentration at which to reformulate a protein in a protein solution that exhibits liquid-liquid phase separation so as to reduce or prevent liquid-liquid phase separation of the protein solution at a desired temperature, comprising:
(a) allowing the protein solution that exhibits liquid-liquid phase separation to undergo liquid-liquid phase separation into an upper and lower liquid phase;
(b) measuring the concentration of the protein in the upper and lower phase of the protein solution that has undergone liquid-liquid phase separation; and
(c) choosing a protein concentration that is greater than the protein concentration of the lower phase to reformulate the protein so as to reduce or prevent liquid-liquid phase separation in the protein solution at the desired temperature.

2. The method of claim 1, wherein the protein is selected from the group consisting of a receptor, a ligand, a transcription factor, an enzyme, and an antibody.

3. The method of claim 1, wherein the protein is a polyclonal antibody or an antigen-binding fragment thereof.

4. The method of claim 1, wherein the protein is a monoclonal antibody or an antigen-binding fragment thereof.

5. The method of claim 1, wherein the desired temperature is the temperature at which the protein solution is to be processed, manufactured, stored, or administered.

6. The method of claim 1, wherein the protein concentration at which to reformulate the protein in the protein solution is chosen to be about 5% to about 40% higher than the concentration of the protein in the lower phase.

7. The method of claim 1, wherein liquid-liquid phase separation is achieved by gravity separation or centrifugation.

8. A method for determining a protein concentration at which to reformulate a protein in a protein solution that exhibits liquid-liquid phase separation so as to reduce or prevent liquid-liquid phase separation of the protein solution at a desired temperature, comprising:
(a) constructing a phase separation curve for the protein solution; and
(b) choosing a concentration that is outside the phase separation curve at the desired temperature,
wherein a concentration that is outside the phase separation curve at the desired temperature is the concentration at which to reformulate the protein in the protein solution so as to reduce or prevent liquid-liquid phase separation in the protein solution at the desired temperature.

9. The method of claim 8, wherein the protein is selected from the group consisting of a receptor, a ligand, a transcription factor, an enzyme, and an antibody.

10. The method of claim 8, wherein the protein is a polyclonal antibody or an antigen-binding fragment thereof.

11. The method of claim 8, wherein the protein is a monoclonal antibody or an antigen-binding fragment thereof.

12. The method of claim 8, wherein the desired temperature is the temperature at which the protein solution is to be processed, manufactured, stored, or administered.

13. The method of claim 8, wherein the protein concentration at which to reformulate the protein in the protein solution is chosen to be about 5% to about 40% higher than the concentration of the protein in the lower phase.

14. The method of claim 8, wherein liquid-liquid phase separation is achieved by gravity separation or centrifugation.

15. A method for formulating a protein in a protein solution so that the protein solution does not exhibit liquid-liquid phase separation at a desired temperature, comprising formulating the protein in the protein solution at a concentration that is outside a phase separation curve for the protein solution at the desired temperature.

16. A method for obtaining a higher concentrating protein solution from a lower concentration protein solution that exhibits liquid-liquid phase separation, comprising:
(a) allowing the original protein solution to separate into an upper and lower phase; and
(b) separating the lower phase from the upper phase,
wherein the lower phase is a protein solution having a higher concentration of the protein than the lower concentration protein solution.

17. The method of claim 16, further comprising adding between about 0.5 mM to about 25 mM calcium chloride or magnesium chloride to the lower phase.

18. The method of claim 16, further comprising adding between about 1 mM to about 145 mM methionine to the lower phase.

19. The method of claim 16, further comprising adding between about 0.5 mM to about 25 mM calcium chloride or magnesium chloride, and between about 1 mM to about 145 mM methionine to the lower phase.
